(19) United States
(12) Patent Application Publication
(10) Pub. No.: US 2003/0060787 A1
(43) Pub. Date: Mar. 27, 2003

(54) Kidney stone collection device and method

(76) Inventors: Cu Ngoc Phan, Newport Coast, CA (US); Khiem Thanh Tran, Newport Coast, CA (US)

Correspondence Address: HAYNES BEFFEL & WOLFELD LLP, P O BOX 366, HALF MOON BAY, CA, 94019, US

(21) Appl. No.: 10/217,697

(22) Filed: Aug. 13, 2002

Related U.S. Application Data
(60) Provisional application No. 60/324,256, filed on Sep. 24, 2001.

(51) Int. Cl. A61F 5/44; A61K 35/23

(52) U.S. Cl. 604/355; 424/558

(57) ABSTRACT

A kidney stone collection device includes entrance and settling chambers coupled at a junction, an inlet opening into the entrance chamber, and an outlet conduit including the device outlet. The outlet conduit also has outlet conduit entrance which opens into the settling chamber. The device includes a base defining the bottom of the settling chamber when the body is in an upright orientation. The outlet conduit entrance is at a higher elevation than at least a portion of the base when the body is in the upright orientation. The body may be made of a pliable and/or transparent material so that the user may see and/or feel kidney stones collected at the base. A shape-maintaining frame element may be used at the inlet to permit the inlet to be maintained in an open configuration.

### CROSS REFERENCE TO RELATED APPLICATION

This application claims the benefit of provisional patent application No. 60/______ filed on ______ Sep. 2001 and entitled Device/Apparatus and Method to Collect Kidney Stones or Stone Fragments from Urine.

### BACKGROUND OF THE INVENTION

The presence of upper urinary tract stones, commonly referred to as kidney stones, is a common medical problem. Fortunately, kidney stones can be effectively treated with extracorporeal shockwave lithotripsy. This is a relatively noninvasive technique during which kidney stones are fragmented by shockwaves applied to the skin surface. The stone fragments can then be passed along with urine via urination. Very small stones may be passed spontaneously without lithotripsy. However, it is a very important to collect the kidney stones and stone fragments for analysis and so that the physician can have the composition determined and then prescribe appropriate medication or other treatment to prevent stone recurrence. Stones and stone fragments will usually be referred to collectively as stones.

FIGS. 1 and 2 illustrate two different devices 10, 12 currently used to collect stones. Collection device 10 is a conical device typically having a paper or plastic body 14 with a net or mesh 16, or other filtering medium, at the lower end. Device 12 is a telescoping, collapsible-cup type device having a net or mesh 18 at the bottom of the device. The body 20 of device 12 is typically made of plastic. These conventional devices work on the filtration principle. Urine is introduced into the device and the net or mesh, or other filtering medium, is supposed to catch the stones. However these devices have some disadvantages. It is sometimes difficult to determine if there are stones or stone fragments caught by the filtering medium. There are often blood clots in the urine of patients with kidney stones. These blood clots may cover stone fragments. The patient may have to use his or her fingers to squeeze the blood clots to see if there stone fragments inside them, an unpleasant task. Very small stone fragments may pass through the filtering medium. In addition, very small stone fragments are often difficult to remove from the filtering medium. The device may be large and inconvenient to carry around; this may discourage patients from collecting stones when away from home. The device may also be relatively expensive.

See, for example, the following the U.S. Pat. Nos.: 3,345,980; 3,774,455; 5,137,031; 5,622,183; 5,762,071; and 5,772,644.

### SUMMARY OF THE INVENTION

The present invention is directed to a kidney stone collection device and method. The device can be made to be inexpensive, compact and easy to carry, and easy to use. The invention enables the user to easily determine if there are any stones in the device from outside the device. The invention is effective at trapping very small stones and stone fragments. It is especially useful for use away from home. The invention can be designed for a single use application or it may be designed to be cleaned and reused.

A first aspect of the invention is directed to a kidney stone collection device including a body positionable at an upright orientation and an inclined orientation. The body defines an entrance chamber and a settling chamber fluidly coupled to one another at a junction. At least a portion of the entrance chamber is located at a higher elevation than the settling chamber when the body is in the upright orientation. The body also includes an inlet, opening into the entrance chamber, and an outlet conduit including an outlet. The outlet conduit also has outlet conduit entrance which opens into the settling chamber. The body also has a base which defines the bottom of the settling chamber when the body is in the upright orientation. The outlet conduit entrance is at a higher elevation than at least a portion of the base when the body is in the upright orientation. The body may be made of a pliable and/or transparent material so that the user may see and/or feel kidney stones collected at the base. A shape-maintaining shapeable frame material may be used at the inlet to permit the inlet to be maintained in an open configuration. The body may also include a user-sealable sealing element configured to seal at least a portion of the settling chamber so to permit any kidney stones to be secured therein.

Another aspect of the invention is directed to a method for collecting kidney stones by directing a urine flow into an entrance chamber of a kidney stone collection device, flowing urine from the entrance chamber, through a settling chamber and out an outlet of the device, and inspecting the settling chamber from outside the settling chamber for the presence of kidney stones. The method may be carried out using a kidney stone collection device in which at least a portion of the settling chamber is pliable and/or transparent. The inspecting step may include looking for kidney stones within the settling chamber and/or manipulating the settling chamber to feel for kidney stones collected within the settling chamber.

Other features and advantages of the invention will appear from the following description in which the preferred embodiments are set forth in detail in conjunction with the accompanying drawings.

### BRIEF DESCRIPTION OF THE DRAWINGS

FIGS. 1 and 2 illustrate two different prior art kidney stone collection devices;

FIG. 3 is a front view of a kidney stone collection device made according to the invention;

FIG. 4 illustrates the device of FIG. 3 with the inlet maintained in a half-way open configuration after manipulating a shape-maintaining shapeable frame element;

FIG. 5 illustrates directing a urine flow into the entrance chamber of the device with excess urine exiting the device through the outlet;

FIG. 6 illustrates placing the device in an inclined orientation to drain urine and other flowable material from the device and visually inspecting the bottom of the settling chamber for the presence of stones;

FIG. 7 shows a user physically manipulating the bottom of the settling chamber to sense for the presence of stones by touch;

FIG. 8 illustrates the device of FIG. 3 modified to include a zip-type seal at the inlet;

FIGS. 9 and 10 illustrate a portion of an alternative embodiment of the invention of FIG. 8 in which the zip-type seal is replaced by an adhesive flap;

FIG. 11 illustrates the device of FIG. 3 with a tear line to facilitate the separation of the lower portion of the settling chamber and a zip-type seal located below the tear line; and

FIGS. 12 and 13 illustrate two further alternative embodiments of the invention shown in inclined orientations.

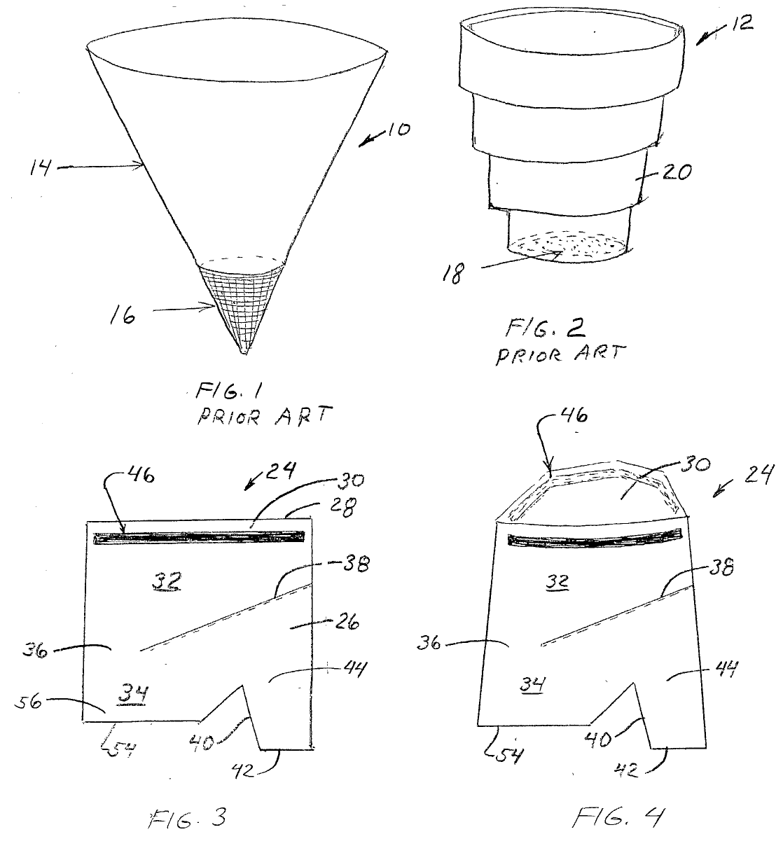

### DETAILED DESCRIPTION OF THE INVENTION

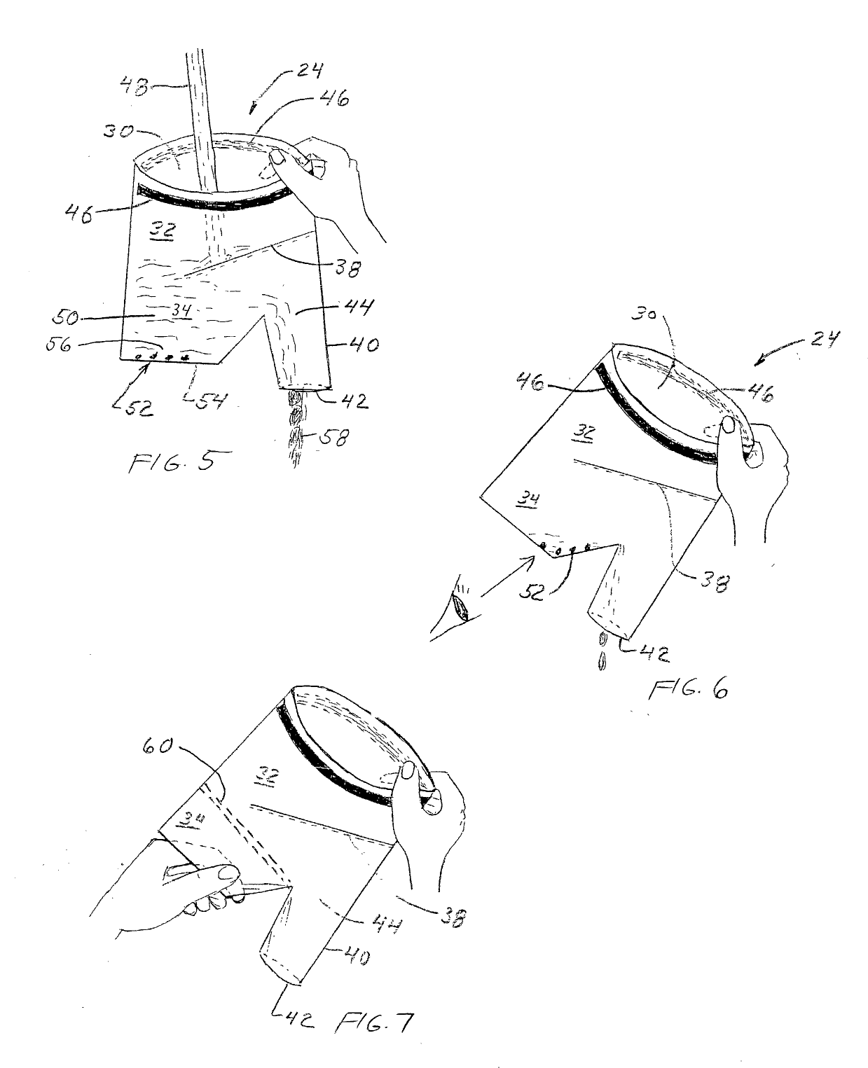

FIGS. 3-7 illustrate a bag-like kidney stone collection device 24 including a body 26 preferably made of a transparent, pliable material such as polyethylene, having an open top 28 which when open defines an inlet 30 to an entrance chamber 32. Body 26 also defines the settling chamber 34 situated below entrance chamber 32 when device 24 is in the upright configuration of FIG. 3. Entrance chamber 32 and settling chamber 34 are fluidly coupled at a junction 36 and are separated by a barrier 38. Barrier 38 partially defines entrance chamber 32, a portion of barrier 38 being located directly vertically above a portion of settling chamber 34 when device 24 is in the upright configuration. Body 26 also includes an outlet conduit 40. Outlet conduit 40 fluidly couples an outlet 42 of device 24 to settling chamber 34 through an outlet conduit entrance 44.

Device 24 also includes a shape-maintaining frame element 46 at inlet 28. Frame element 46 is made of metal or other shapeable, shape-maintaining material which permits a user to open top 28 and to maintain inlet 30 into entrance chamber 32 in an open condition as a urine stream 48 is directed into entrance chamber 32 as shown in FIG. 5. This aids 1-handed manipulation of device 26 as urine stream 48 is directed into chamber 32. Inlet 30, when open, preferably has a cross-sectional dimension of about 9 cm (3.5 inches). Urine stream 48 may be a provided by urination directly into entrance chamber 32 or may be provided from a urine container. Urine stream 48 is typically partially deflected by impact against barrier 38, after which urine 50 passes into settling chamber 34. Stones 52, that is kidney stones and kidney stone fragments, collect along the base 54 of body 26, base 54 at least partially defining the bottom 56 of settling chamber 34. Barrier 38 is preferably positioned so that it overlies that portion of bottom 56 of settling chamber 34 adjacent to outlet conduit entrance 44; this helps prevent stones 52 from being ejected through outlet conduit entrance 44 by the force of urine stream 48.

Excess urine 58 passes through settling chamber 34, through outlet conduit entrance 44, along outlet conduit 40 and exits device 24 through outlet 42. This continuous diversion of excess urine 58 permits device 24 to be relatively small and yet be capable of capturing all, or at least substantially all, stones 52 from urine flow 48. Base 54 is located at a higher elevation than outlet 42 and is laterally offset from outlet 42 when device 24 is in the upright configuration. This helps to prevent excess urine 58 from contaminating the outside of device 24.

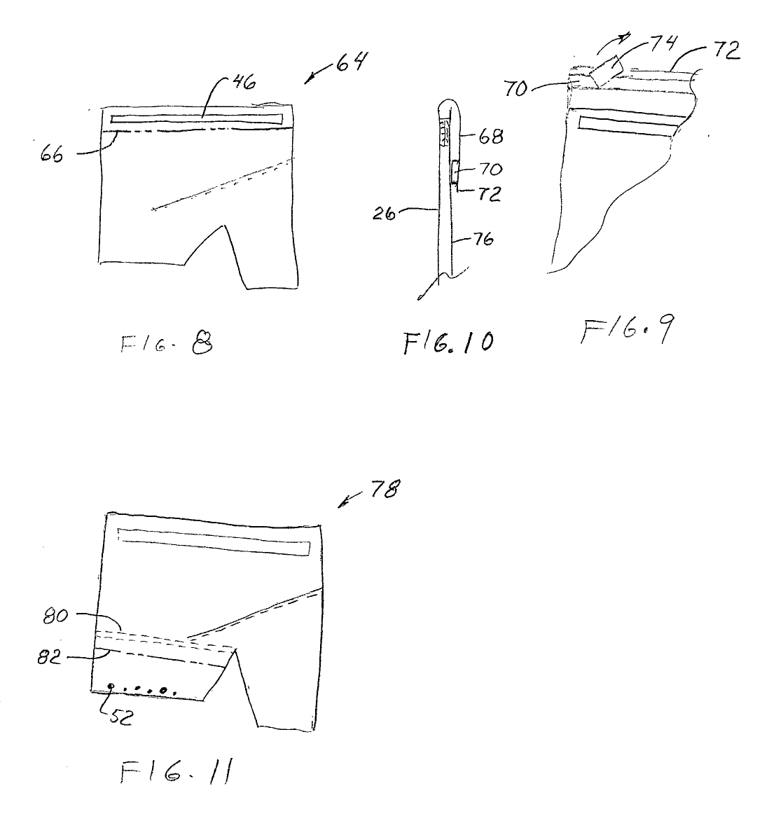

After the introduction of urine into entrance chamber 32 has been completed, device 24 may be tilted to the inclined condition as shown in FIG. 6 to remove substantially all of the urine within settling chamber 34. Blood clots will tend to flow out of device 24 with excess urine 58. Before, during or after this process the user can visually inspect settling chamber 34 for the presence of stones 52 because of the transparent nature of body 26. If desired, only the portion of body 26 at which stones 52 collect could be made to be transparent, or at least translucent. In addition, the presence of stones 52 can be determined by physically manipulating at least a portion of settling chamber 34 to sense the presence of stones by touch. See FIG. 7. Water or other liquid could be used to help clean the stones prior to removal from device 24.

Access to stones 52 may be aided by provision of a weakened region 60, indicated by double dashed lines in FIG. 7, to permit body 26 to be torn along weakened region 26. Instead of removing stones 52, it may be desired to retain stones 52 within settling chamber 34. In such a case the user may simply fold device 24, typically using multiple folds, and place the folded device into a sealable plastic bag or other stone container.

Device 24 could also be adapted to permit it to be sealed with stones 52 therein. For example, FIG. 8 illustrates a stone collection device 64 similar to device 24 but including a zip-type seal 66 at inlet 30 and adjacent to frame element 46. FIGS. 9 and 10 illustrate a further embodiment in which a flap 68 extends from one side of body 26 and has an adhesive strip 70 along its outer edge 72. Adhesive strip 70 is covered by a release film 74; the release film is shown being removed in FIG. 9. To secure stones 52 within the device, release film 74 is removed and flap 68 is folded down over top 28 of body 26 to permit adhesive strip 70 to adhere to the front surface 76 of body 26 and effectively seal inlet 30. FIG. 11 illustrates a further embodiment of a stone collection device 78. Device 78 is similar to the collection devices illustrated in FIG. 7 and 8 and includes a weakened region 80 and a zip-type seal 82 located adjacent to weakened region 80. After removing excess urine, seal 82 may be closed and that portion of settling chamber 34 containing stones 52 may be separated from the remainder of the device along weakened region 80.

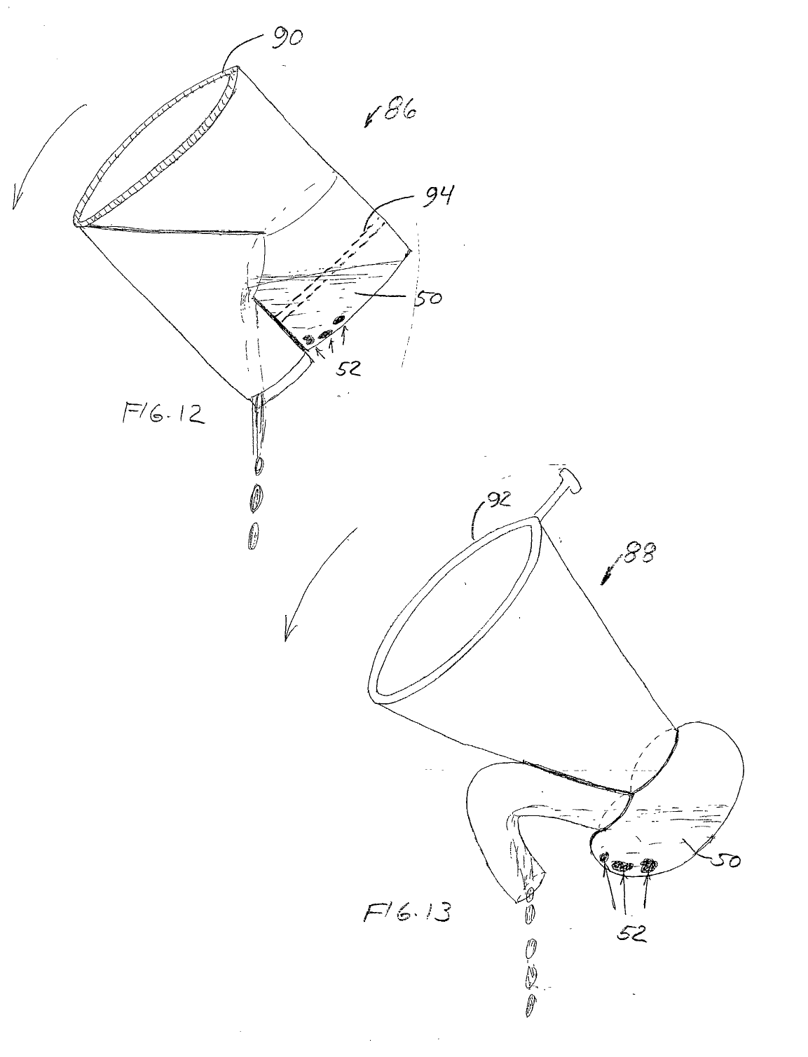

FIGS. 12 and 13 illustrate two alternative, funnel-type embodiments of the invention. Stone collection devices 86, 88 are shown in their inclined orientations draining excess urine from their settling chambers leaving stones along the bases of the settling chambers. While devices 86, 88 may be made of rigid material, they are preferably made of pliable, transparent material having an attached frame/handle 90, 92 along each of their tops. Frame/handles 90, 92 may be rigid or may be a shape-maintaining element, typically made of metal or plastic. Stone collection device 86 is illustrated to include a weakened region 94 to facilitate removal of stones 52.

Modification and variation can be made to be disclosed embodiments and methods without departing from the subject of the invention as defined in the following claims. For example, frame element 46 could be made of rigid elements designed to be temporarily manipulated into or secured in an open configuration to keep inlet 30 open while urine stream 48 is directed into entrance chamber 32. Frame element 46 could also be a spring-type shape-maintaining frame element which when released from a closed configuration assumes an open configuration to open inlet 30. While the presence of stones 52 is preferably sensed by both sight and touch, and in some situations in may be desirable to design the collection device so to permit detection of the presence of stones by only sight (e.g., when body 26 is transparent but made of a relatively thick and stiff material) or only by touch (e.g., when body 26 is opaque but made of a thin, pliable material).

Any and all patents, patent applications and printed publications referred to above are incorporated by reference.

What is claimed is:

1. A kidney stone collection device comprising:
a body positionable in an upright orientation and an inclined orientation;
said body defining an entrance chamber and a settling chamber, at least a portion of the entrance chamber located at a higher elevation than the settling chamber when the body is in the upright orientation;
the entrance and settling chambers fluidly coupled to one another at a junction;
the body comprising:
an inlet, opening into the entrance chamber, and an outlet;
an outlet conduit comprising said outlet and also comprising an outlet conduit entrance, said outlet conduit entrance opening into the settling chamber; and
a base defining a bottom of the settling chamber when the body is in the upright orientation; and
the outlet conduit entrance being at a higher elevation than at least a portion of the base when the body is in the upright orientation.

2. The device according to claim 1 wherein the body comprises a barrier partially defining the entrance chamber, at least a portion of the barrier extending directly vertically above at least a portion of the settling chamber when the body is in the upright orientation.

3. The device according to claim 1 wherein the body comprises a transparent material.

4. The device according to claim 1 wherein at least a portion of the body at the base is a transparent material, whereby the user may see kidney stones collected at the base.

5. The device according to claim 1 wherein at least a portion of the body at the base is a pliable material, whereby the user may feel kidney stones collected at the base.

6. The device according to claim 1 wherein at least a portion of the body at the base is a pliable, transparent material, whereby the user may see and feel kidney stones collected at the base.

7. The device according to claim 1 wherein the body comprises a pliable bag-like material, and a further comprising a shape-maintaining frame element at the inlet so to aid maintaining the inlet in an open configuration.

8. The device according to claim 1 further comprising a shape-maintaining frame element at the inlet so to aid maintaining the inlet in an open configuration.

9. The device according to claim 8 wherein the frame element is made of metal.

10. The device according to claim 1 wherein the entire entrance chamber is located at a higher elevation than the settling chamber when the body is in the upright orientation.

11. The device according to claim 1 wherein the base is at a higher elevation than the outlet when the body is in the upright orientation.

12. The device according to claim 1 wherein the outlet is laterally spaced-apart from the base when the body is in the upright orientation.

13. The device according to claim 1 wherein the junction and the outlet conduit entrance are at different elevations when the body is in the upright orientation.

14. The device according to claim 1 wherein the body further comprises a user-sealable sealing element configured to seal least a portion of the device so to permit any kidney stones to be secured therein.

15. The device according to claim 14 wherein the sealing element is at the inlet.

16. The device according to claim 1 wherein the body comprises a weakened region to aid separating at least a portion of the device from the remainder of the device.

17. A kidney stone collection device comprising:
a body positionable in an upright orientation and an inclined orientation;
said body defining an entrance chamber and a settling chamber, at least a majority of the entrance chamber located at a higher elevation than the settling chamber when the body is in the upright orientation;
the entrance and settling chambers fluidly coupled to one another at a junction;
the body comprising:
an inlet, opening into the entrance chamber, and an outlet;
an outlet conduit comprising said outlet and also comprising an outlet conduit entrance, said outlet conduit entrance opening into the settling chamber;
a base defining a bottom of the settling chamber when the body is in the upright orientation;
a barrier partially defining the entrance chamber, at least a portion of the barrier extending directly vertically above at least a portion of the settling chamber when the body is in the upright orientation; and
a transparent, pliable bag-like material whereby the user may see and feel kidney stones collected at the base;
a shape-maintaining frame element at the inlet so to aid maintaining the inlet in an open configuration; and
the outlet conduit entrance being at a higher elevation than at least a portion of the base when the body is in the upright orientation.

18. The device according to claim 17 wherein the body comprises a user-sealable sealing element configured to seal least a portion of the device so to permit any kidney stones to be secured therein.

19. The device according to claim 17 wherein the body comprises a weakened region to aid separating at least a portion of the device from the remainder of the device.

20. A method for collecting kidney stones comprising:
directing a urine flow into an entrance chamber of a kidney stone collection device, said device comprising an outlet and a settling chamber between the entrance chamber and the outlet;
flowing urine from the entrance chamber, through the settling chamber and out the outlet; and
inspecting the settling chamber from outside the device for the presence of kidney stones.

21. The method according to claim 20 further comprising selecting a kidney stone collection device in which at least a portion of the settling chamber is pliable and transparent.

22. The method according to claim 21 wherein the inspecting step is carried out by looking for kidney stones within the settling chamber and manipulating the settling chamber to feel for kidney stones collected within the settling chamber.

23. The method according to claim 20 wherein the inspecting step is carried out by visually inspecting for stones within at least a portion of the settling chamber.

24. The method according to claim 20 wherein the inspecting step is carried out by physically manipulating at least a portion of the settling chamber to sense the presence of kidney stones by touch.

25. The method according to claim 20 further comprising selecting a bag-like kidney stone collection device.

26. The method according to claim 25 further comprising maintaining an inlet to the entrance chamber in an open condition using a shape-maintaining frame element at least partially surrounding the inlet.

27. The method according to claim 20 wherein the urine flowing step comprises:
permitting urine to flow through the entrance and settling chambers and out the outlet during the urine flow directing step; and after the urine directing step
draining at least a portion of any urine remaining in the device.

28. The method according to claim 27 wherein the urine flow permitting step is carried out with the device in a generally upright orientation.

29. The method according to claim 27 wherein the draining step is carried out with the device in an inclined orientation.

30. The method according to claim 20 further comprising passing a fluid through the device to remove urine and other flowable material while leaving any collected kidney stones within the settling chamber.

31. The method according to claim 20 further comprising removing any collected kidney stones from the device for analysis.

32. The method according to claim 20 further comprising:
selecting a foldable kidney stone collecting device; and
if the inspecting step indicates the presence of kidney stones, folding the kidney stone collecting device to secure the collected kidney stones therein.

33. The method according to claim 20 further comprising:
selecting a sealable kidney stone collecting device; and
if the inspecting step indicates the presence of kidney stones, sealing at least a portion of the device to secure the collected kidney stones therein.

34. The method according to claim 33 wherein the sealing step is carried out by sealing the entrance chamber at the inlet.

35. A method for collecting kidney stones comprising:
selecting a bag-like kidney stone collection device, comprising an entrance chamber, an outlet and a settling chamber between the entrance chamber and the outlet, at least a portion of said settling chamber being pliable and transparent to permit a user to see and feel kidney stones collected within the settling chamber;
directing a urine flow into the entrance chamber of the device;
maintaining an inlet to the entrance chamber in an open condition using a shape-maintaining frame element at least partially surrounding the inlet of the device;
flowing urine from the entrance chamber, through the settling chamber and out the outlet, with the device in a generally upright orientation;
draining at least a portion of any urine remaining in the device, with the device in an inclined orientation;
passing a fluid through the device to remove urine and other flowable material while leaving any collected kidney stones within the settling chamber; and
inspecting the settling chamber from outside the device for the presence of kidney stones.

36. The method according to claim 35 wherein the inspecting step is carried out by visually inspecting at least a portion of the settling chamber.

37. The method according to claim 35 wherein the inspecting step is carried out by physically manipulating at least a portion of the settling chamber to sense the presence of kidney stones by touch.

38. The method according to claim 35 further comprising removing any collected kidney stones from the device for analysis.

39. The method according to claim 35 wherein if the inspecting step indicates the presence of kidney stones, folding the bag-like kidney stone collecting device to secure the collected kidney stones therein.

40. The method according to claim 35 wherein if the inspecting step indicates the presence of kidney stones, sealing at least a part of the device to secure the collected kidney stones therein.
